(19) United States
(12) Patent Application Publication
(10) Pub. No.: US 2026/0248975 A1
(43) Pub. Date: Aug. 27, 2026

(54) DEVICE FOR EMITTING CLEANSING GASSES

(71) Applicant: Joshua IGERT, Henderson, NV (US)

(72) Inventor: Joshua IGERT, Henderson, NV (US)

(21) Appl. No.: 19/549,745

(22) Filed: Feb. 25, 2026

Related U.S. Application Data
(60) Provisional application No. 63/762,928, filed on Feb. 25, 2025.

(51) Int. Cl. A61L 2/22; A61L 9/012; A61L 103/97

(52) U.S. Cl. CPC A61L 2/22; A61L 9/012; A61L 2103/97; A61L 2202/11; A61L 2202/15; A61L 2209/14; A61L 2209/21

(57) ABSTRACT

A device for emitting cleaning gasses into a space includes a vessel extending about and along an axis and having a base and a sidewall extending between a lower end and an upper end to define a chamber. A pump is configured to generate pressurized air. An air emitting device is disposed in the chamber adjacent to the base and is fluidly connected to the pump. During operation, the pump delivers pressurized air through the air emitting device to aerate a cleaning chemical within the chamber and thereby release cleaning gasses into the space.

### CROSS-REFERENCE TO RELATED APPLICATIONS

The present application claims priority to U.S. Provisional Patent Application Ser. No. 63/762,928 filed Feb. 25, 2025 and entitled “DEVICE FOR EMITTING CLEANSING GASSES,” the entirety of which is incorporated by reference herein.

### FIELD OF THE DISCLOSURE

The present disclosure relates to cleaning devices. More particularly, the present disclosure relates to devices for emitting disinfecting gasses to sanitize contaminated spaces and eliminate odors.

### BACKGROUND

Frequently used spaces, like the interiors of vehicles, are prone to accumulating dirt, grime, microbes, bacteria, etc. which can lead to an overall unsanitary environment and unpleasant odors. Factors such as smoking, exposure to ill passengers, exposure to the outdoors and the presence of pets can contribute to the creation of an unsanitary environment. Cleaning gas emitting vessels are known for disinfecting such spaces. Cleaning vessels typically include an agitating component like an impeller in the vessel to mechanically agitate a mixture of chlorine dioxide (typically from a dissolving tablet) and water to release cleaning gasses into the environment. An issue with such cleaning vessels is that the impeller cannot be activated until the tablet has fully dissolved. There remains a need for improvements to such cleaning devices.

### SUMMARY OF THE DISCLOSURE

According to an aspect of the disclosure, a device for emitting cleaning gasses into a space includes a vessel extending about and along an axis and having a sidewall extending between a lower end and an upper end and having a base adjacent to the lower end and defining a chamber. A pump is configured to emit a pressurized air. An air emitting device is located in the chamber of the vessel adjacent to the base and fluidly connected to the pump for emitting pressurized air from the pump into the chamber to aerate a cleaning chemical in the chamber to release cleaning gasses into the space.

According to another aspect of the disclosure, a method for emitting cleaning gasses into a space including positioning a vessel in the space. The vessel extends about and along an axis and has a base and a sidewall extending between a lower end and an upper end. The vessel defines a chamber. The method further includes placing a cleaning chemical in the chamber. The method also includes operating a pump to deliver pressurized air through an air emitting device located in the chamber adjacent to the base to aerate the cleaning chemical and release cleaning gasses into the space.

The use of an air emitting device provides a very quick manner of releasing gasses from the cleaning chemical, thus providing a fast and efficient means for eliminating odors and sanitizing the space. The subject device advantageously emits gasses even while chemical tablets are in the process of dissolving and can be used to emit gasses from chemicals at various concentrations.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is a perspective view of a first embodiment of a device for emitting cleaning gasses according to an aspect of the disclosure;

FIG. 2 is a top view of a pump housing of the first embodiment of the device for emitting cleaning gasses;

FIG. 3 is a perspective view of the pump housing of the first embodiment;

FIG. 4 is a perspective view of a vessel of the first embodiment of the device for emitting cleaning gasses;

FIG. 5 is a top view of the vessel of the first embodiment;

FIG. 6 is a bottom view of the vessel of the first embodiment;

FIG. 7 is a perspective, bottom view of the vessel of the first embodiment;

FIG. 8 is a side, perspective view of the vessel of the first embodiment, illustrating a pump connected to a bottom face of the vessel;

FIG. 9 is a top view of the vessel of the first embodiment, illustrating an air emitting device located along the base of the vessel;

FIG. 10 is a top view of the vessel of the first embodiment, illustrating the air emitting device agitating a cleaning chemical mixture in the vessel;

FIG. 11 is a perspective view of a lid of the first embodiment of the device for emitting cleaning gasses;

FIG. 12 is a bottom view of the lid of the first embodiment;

FIG. 13 is a side, perspective, exploded view of a second embodiment of the device for emitting cleaning gasses;

FIG. 14 is a another side, perspective, exploded view of the second embodiment of the device for emitting cleaning gasses;

FIG. 15 is a side, perspective view of the second embodiment of the device for emitting cleaning gasses;

FIG. 16 is a top, perspective view of a pump housing of the second embodiment, illustrating removal of a pump cap;

FIG. 17 is a bottom view of the pump housing of the second embodiment;

FIG. 18 is a bottom view of the pump cap of the second embodiment;

FIG. 19 is a top view of the pump cap of the second embodiment;

FIG. 20 is a side, perspective view of a connection between a vessel and a pump housing of the second embodiment;

FIG. 21 is a bottom view of a filter housing of the second embodiment;

FIG. 22 is a bottom view of the filter housing of the second embodiment, illustrating a filter received by the filter housing;

FIG. 23 is a side, perspective view of an inner sleeve of a vessel of a third embodiment of the device for emitting cleaning gasses;

FIG. 24 is a top, perspective view of the inner sleeve of the vessel of the third embodiment;

FIG. 25 is a bottom, perspective view of the inner sleeve of the vessel of the third embodiment; and

FIG. 26 is a side, perspective view of the inner sleeve and an outer sleeve of the vessel of the third embodiment.

### DESCRIPTION OF THE ENABLING EMBODIMENTS

Example embodiments of a device 10, 110 for emitting cleaning gasses of the present disclosure will now be described more fully with reference to the accompanying drawings. However, the example embodiments are only provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that the example embodiments may be embodied in many different forms and that neither should be construed to limit the scope of the disclosure. Features of the embodiments may be combined with one another. In some example embodiments, well-known processes, well-known device structures, and well-known technologies are not described in detail.

Referring to the figures, wherein like numerals indicate corresponding parts throughout the several views, embodiments of the device 10, 110 for emitting cleaning gasses, such as Chlorine Dioxide, into a space are shown. Other cleaning chemicals may include ozone, hydrogen peroxide vapor, chlorine gas, hypochlorous acid vapor, sulfur dioxide, nitrogen dioxide (and other nitrogen oxides), formaldehyde, ethylene oxide, and the like. The chemicals may be in the form of tablets, a two-part powder system, premixed liquids, gels, or in other forms. According to a preferred embodiment, the device 10, 110 is used to clean and eliminate odors from the interior of a vehicle, such as an automobile, but could be used to clean and eliminate odors from various other spaces such as hospital rooms, hotel rooms, trains, buses, and the like. The device 10, 110 may be used to eliminate odors that may result from contaminants like cigarette smoke as well as other varieties of stench causing malodors like mold bacteria mildew. Another purpose of the device 10, 110 is to eliminate bedbugs from interior spaces like hotel rooms.

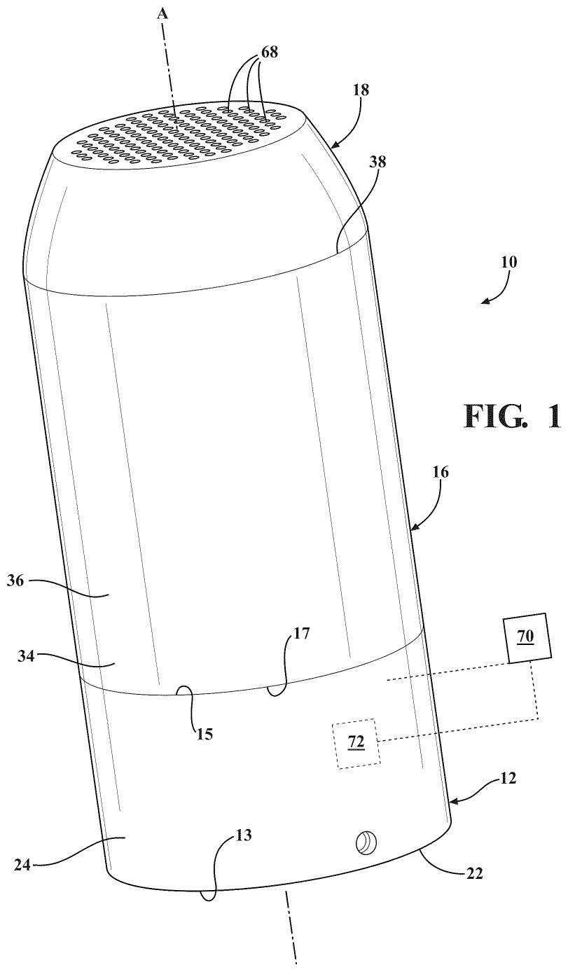

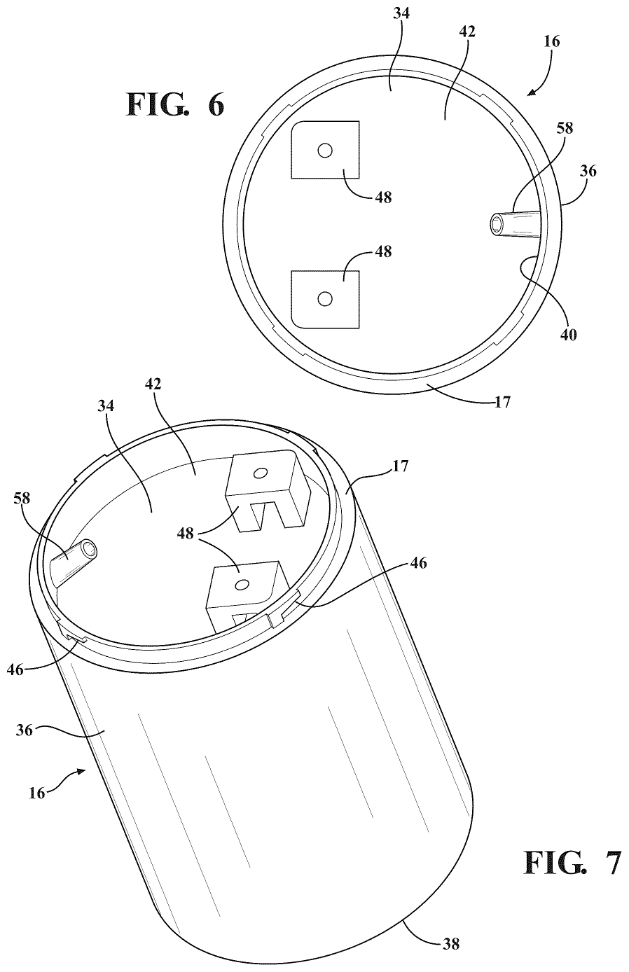

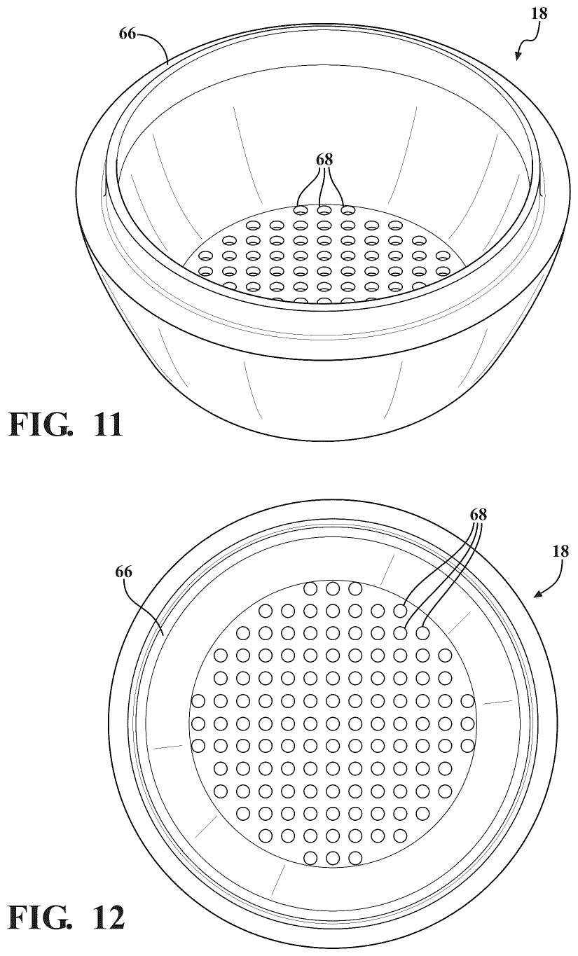

According to a first embodiment of the device 10 shown in FIGS. 1-12, the device 10 generally includes a pump housing 12 that contains a pump 14, a vessel 16 that is coupled with the pump housing 12 above the pump housing 12, and a lid 18 which closes a top of the vessel 16. An air emitting device 20 is located in the vessel 16 for agitating a chemical mixture in the vessel 16 to efficiently emit cleaning gasses into the surrounding space to disinfect the surrounding space.

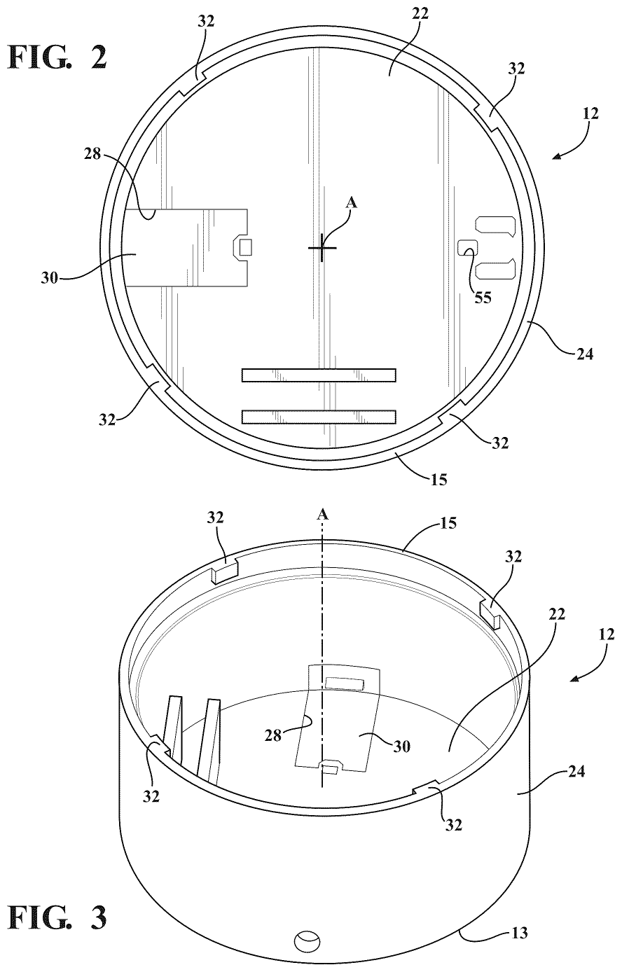

As best shown in FIGS. 2-3, the pump housing 12 generally has a cup shape and has a cylindrical wall 24 that extends axially between a bottom end 13 and a top end 15. A bottom segment 22 is located along, and closes the bottom end 13. A compartment 26 is located above the bottom segment 22 internally of the cylindrical wall 24. As discussed in further detail below, the compartment 26 is configured to hold the pump 14 as well as other electronic components, e.g., a controller 70, in a sealed manner which prevents contaminants from disrupting operation of the pump 14 and controller 70. The bottom segment 22 defines a recess 28 that removably contains one or more batteries for powering the pump 14. The bottom segment 22 also includes a battery cover 30 that detachably closes the recess 28 for holding and sealing the battery in place. A plurality of tabs 32 are positioned along the top end 15 of the cylindrical wall 24 in circumferentially spaced relationship with one another.

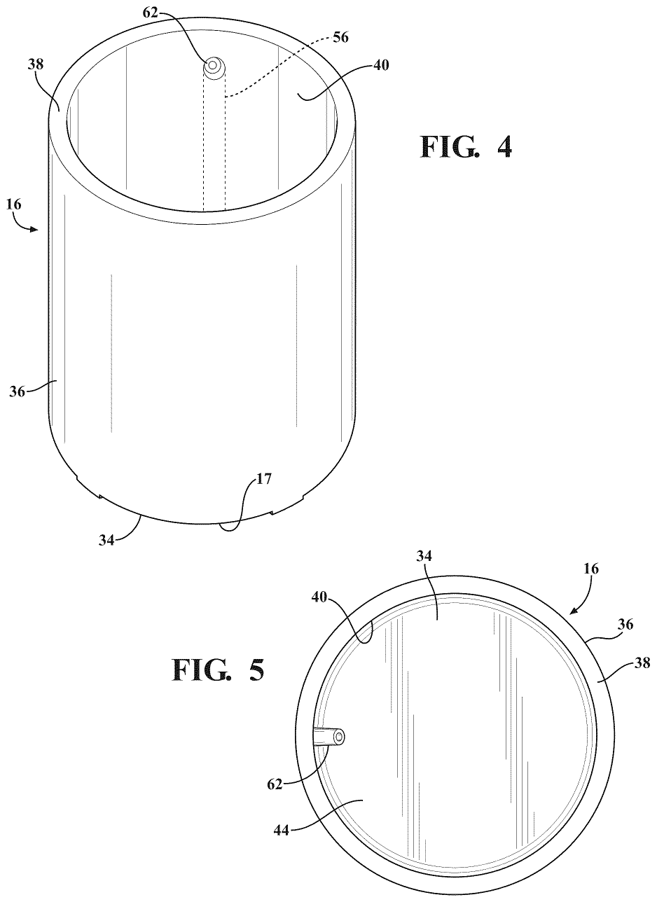

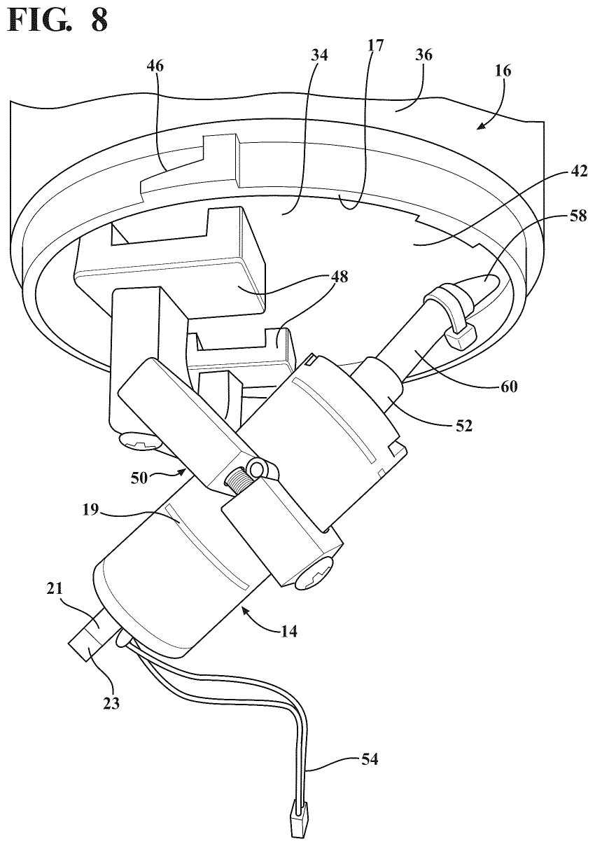

As shown in FIG. 1, the vessel 16 is coupled to the top of the pump housing 12. With reference to FIGS. 4-10, the vessel 16 has an annular sidewall 36 that extends axially between a lower end 17 and an upper end 38, and has a base 34 along the lower end 17. A compartment 40 is defined between the base 34 and sidewall 36. The base 32 has a bottom face 42 outside of the compartment 40 and a top face 44 in the compartment 40. As best shown in FIGS. 7-8, a perimeter of the base 34 of the vessel 16 defines a plurality of slots 46 for receiving the tabs 32 of the pump housing 12 for releasably coupling the vessel 16 to the pump housing 12 and sealing the chamber 26 of the pump housing 12 upon insertion of tabs 32 into the slots 46 via rotation of the pump housing 12 relative to the vessel 16. It should be appreciated that other fastening mechanisms may be used to sealingly connect the pump housing 12 to the vessel 16, e.g., threads, without departing from the scope of the present disclosure. The ability to quickly and sealingly connect and disconnect the pump housing 12 to the vessel 16 provides easy access to the contents of the pump housing 16, such as for repairs.

As best shown in FIGS. 7-8, a pair of mounting blocks 48 protrude from the bottom face 42 of the vessel 16. The mounting blocks 48 are arranged in spaced and parallel relationship with one another. The pump 14 is coupled to the pair of mounting blocks 48 via a bracket 50 such that the pump 14 is located in the compartment 26 of the pump housing 12 when the pump housing 12 is connected to the bottom face 42 of the vessel 16. The pump 14 has an outlet 52 that is configured to emit pressurized air. An electrical connector 54 electrically couples the pump 14 to the one or more batteries and a controller 70 for powering and selectively controlling the pump 14. The electrical connector 54 may be connectable to a socket 55 on the bottom segment 22 of the pump housing 12. According to the preferred embodiment, the pump 14 is powered via a DC motor, but other styles of pumps 14 could be used without departing from the scope of the subject disclosure. The pump 14 contains a chemical resistant diaphragm 19 (schematically shown in FIG. 8) such as polytetrafluoroethylene (PTFE), tephlon, polyvinyl chloride (PVC), or viton, or any similar chlorine dioxide resistant material inside of the pump, to inhibit deterioration of components. Specially, the diaphragm 19 is located between a fluid chamber and drive mechanism of the pump 14. The diaphragm 19 is configured to flex and move air or fluid while protecting the pump 14 from corrosive cleaning agents. As also schematically shown, the pump 14 has an intake 21 for drawing air into the pump 14. A filter 23 may be located along the intake 21 of the pump 14 to prevent the pump 14 from intaking the chlorine dioxide gas (or other cleaning chemicals) into the pump 14. The diaphragm 19 and filtration system 23 permit the device 10 to continuously operate in environments with extremely high doses of chemicals, including high concentration of chlorine dioxide.

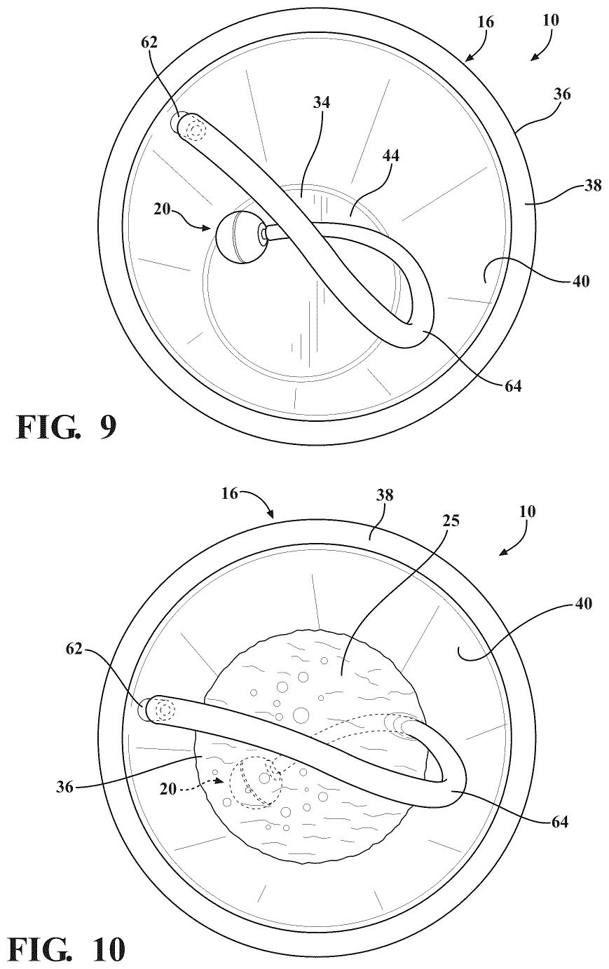

As shown in dashed lines in FIG. 4, an air passage 56 is defined inside the sidewall 36 of the vessel 16 and extends axially upwardly from a location adjacent to the base 34 of the vessel 16 to a location adjacent to the top 38 of the vessel 16. As shown in FIGS. 5-7, an inlet connector 58 extends from the sidewall 36 of the vessel 16 under the bottom face 42 of the base 34 of the vessel 16. The inlet connector 58 is fluidly connected to the air passage 56. A first hose 60 interconnects the inlet connector 58 and the outlet 52 of the pump 14 to permit the pump 14 to pass pressurized air into the air passage 56. As best shown in FIGS. 9-10, an outlet connector 62 extends from the sidewall 36 of the vessel 16 into the chamber 40 from a location adjacent to the top 38 of the vessel 16. A second hose 64 is coupled to the outlet connector 62 and extends downwardly toward the base 34 of the vessel 16.

The air emitting device 20 is connected to the second hose 64 adjacent to/along the base 34 of the vessel 16 for emitting pressurized air from the pump 14 into the chamber 40 of the vessel 16. According to the preferred embodiment, the air emitting device 20 is an air stone 20, but other types of air emitting devices 20 could be used without departing from the scope of the subject disclosure. As will be discussed in further detail below, the air stone 20 is configured to agitate a cleaning chemical mixture 25 in the chamber 40 of the vessel 16 to cause cleaning gasses to be emitted through the lid 18 of the vessel to disinfect the surrounding space.

Routing pressurized air upward through the air passage 56 and then downward through the second hose 64 to the air stone air emitting device 20 generally reduces leakage risks at the chemical chamber 40 by avoiding a pressurized, sealed pass-through through the base 34, while keeping the only wetted delivery components inside the chamber 10 limited to inexpensive, replaceable parts such as the second hose 64 and air emitting device 20. This arrangement also improves chemical isolation of the pump 14, particularly where corrosive agents such as chlorine dioxide are used, by physically separating the compartment of the pump housing 12 from the chemical chamber 40 and reducing liquid ingress and vapor backflow during sloshing or tipping. In addition, where the vessel 16 is removably coupled to the pump housing 12, the sidewall routing simplifies the mechanical interface by avoiding a wet, pressurized joint at the coupling line. The sidewall passage 56 further enhance manufacturability and robustness by being integrally molded, and improves operational cleanliness by reducing clogging risks at the base 34 and allows easy replacement of fouled internal tubing. Finally, the vertical passage 56 and second hose 64 prevent liquids from siphoning back to the pump 14.

With reference to FIGS. 1 and 11-12, the lid 18 is coupled to the top 38 of the vessel 16 via insertion of an annular flange 66 of the lid 18 into the open top 38 of the vessel 16. The lid 19 could be coupled in other ways, e.g., threads, without departing from the scope of the subject disclosure. The lid 18 defines an array of orifices 68 to permit the gasses to escape from the chamber 40 of the vessel 16 while preventing large volumes of liquids from splashing out of the vessel 16.

The controller 70 (schematically shown in FIG. 1) is electrically connected to the pump 14 for activating and deactivating the pump 14. The controller 70 may also be configured to include various operation modes, such as a time of operation and flow rate of air being emitted from the pump 14 to provide a desired cleaning effect. An input mechanism 72 (schematically shown in FIG. 1) is electrically connected to the controller 70 for allowing an operator to activate and deactivate the pump 14. The input mechanism 72 may take the form of one or more physical buttons on the device or may be part of a standalone device like a remote, smart phone, tablet, etc.

A method of using the device 10 includes filling the chamber 40 of the vessel 16 with the chemical 25 alone or with water and activating the pump 14 with the input mechanism 72 to cause the air emitting device 20 to agitate the chemical/water to enhance a dispersal of gasses from the chemical 25 through the lid 18. The pump 14 steadily aerates the mixture, releasing trapped gas into the space, removing odors and sanitizing all surfaces it comes into contact with. The gasses are configured to be emitted throughout the space.

Aeration via the air emitting device 20 expels gasses quicker than prior agitation techniques, such as impellers, thus providing an overall faster sanitizing effect. Furthermore, the subject device 10 may be used while chemical tablets are dissolving, which is not possible with prior device that rely on mechanical rotation of an impeller. Furthermore, the device 10 does not rely on sensors like prior devices and can run with any concentration of cleaning chemicals based on specific needs. Furthermore, the device 10 is configured to emit gasses from the entire volume of the vessel 16, which can be problematic on prior devices in which chemicals can become suspended or trapped in a liquid and ultimately not emitted from the vessel 16. Movement of the gasses may be assisted via means such as fans or an air circulation system of the vehicle.

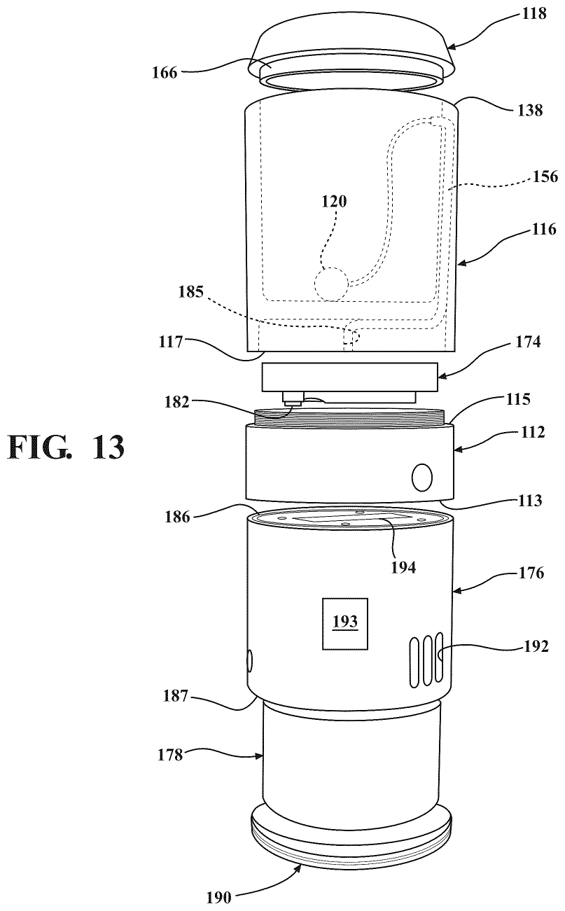

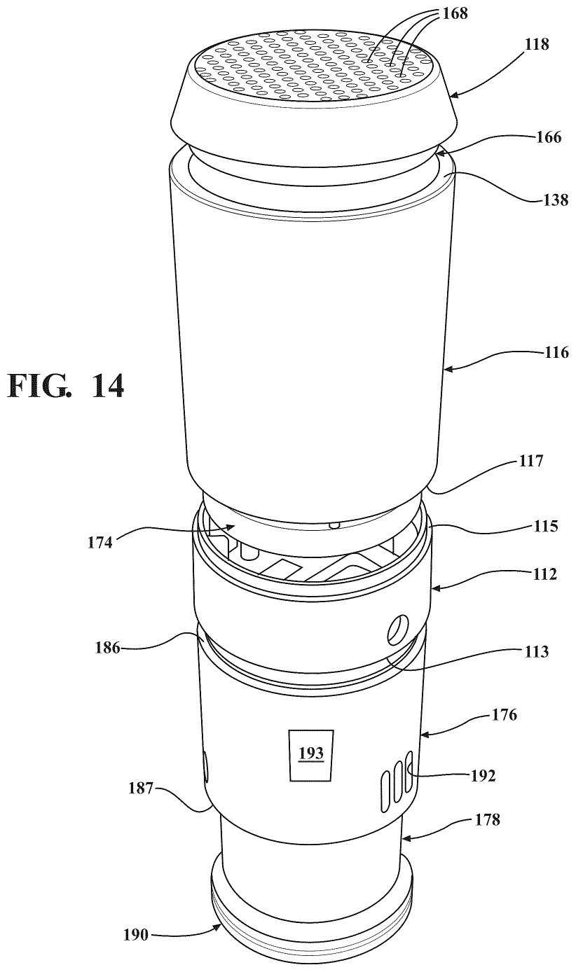

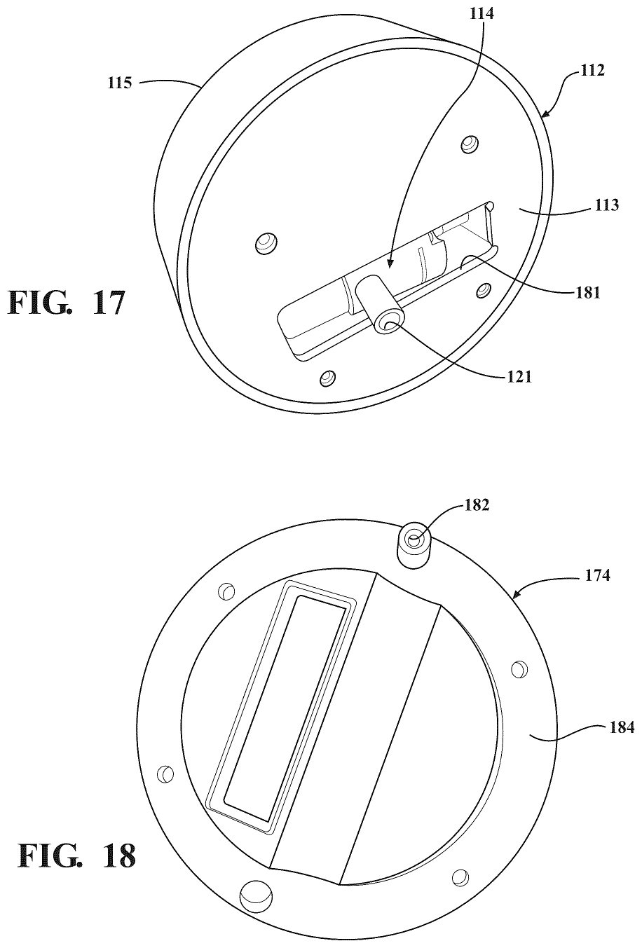

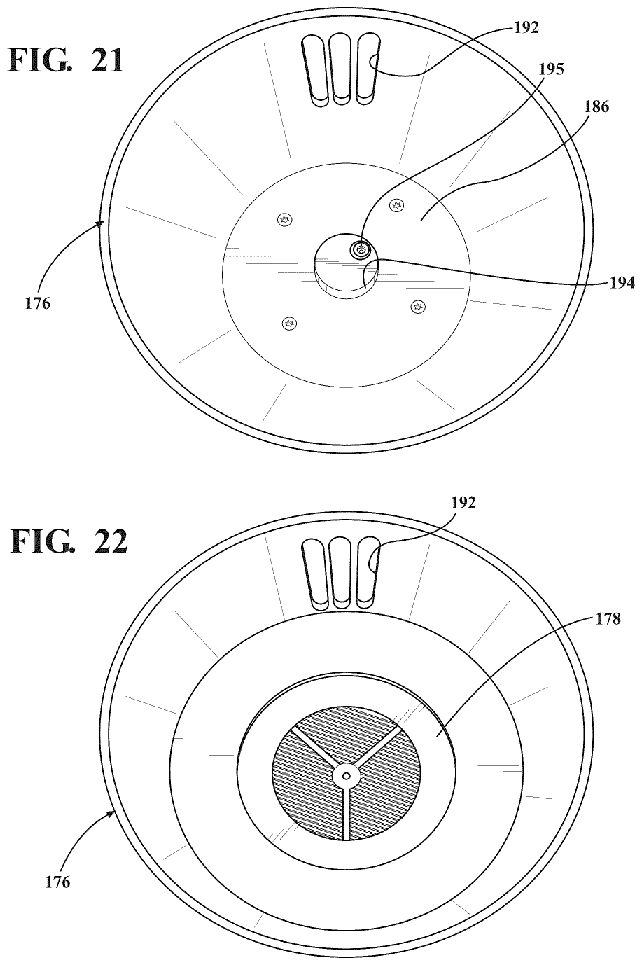

FIGS. 13-22 illustrate a second embodiment of the device 110 for emitting cleaning gasses, wherein like numerals, separated by a prefix of “1” identify corresponding components with the first embodiment described above. Of note, this assembly includes a unique arrangement of a pump housing 112, a pump cap 174, a filter housing 176, a filter 178, and a bottom cap 190.

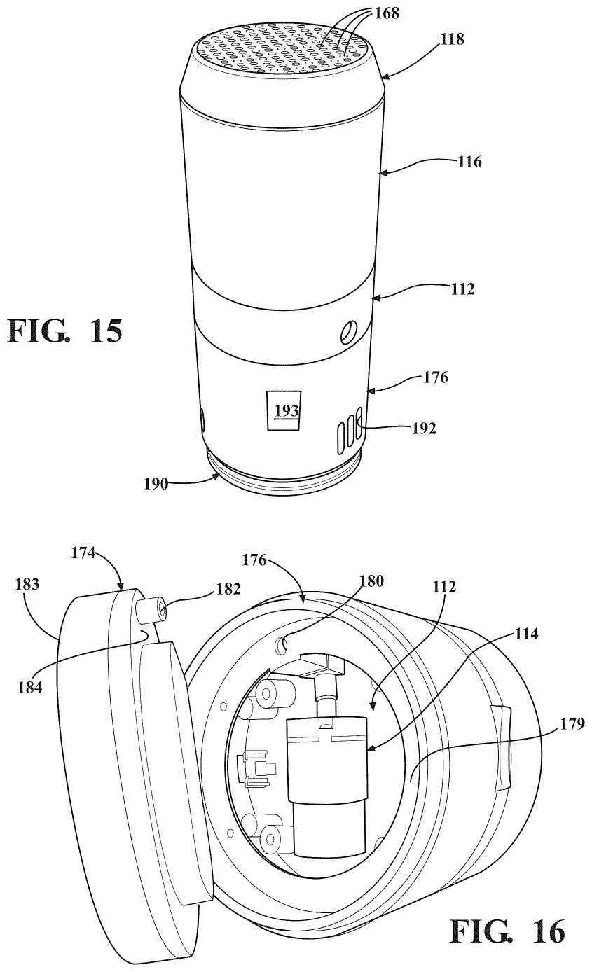

More particularly, a top end 115 of the pump housing 112 is threadedly connectable to the lower end 117 of the vessel 116. It may be connected in other ways. As best shown in FIG. 15, the pump housing 112 contains the pump 114. A flange 179 is located adjacent to the top end 115 of the pump housing 112 which defines a flow channel 80. The outlet 152 of the pump 114 is aligned with the flow channel 80 such that pressurized air from the pump 114 passes through the flow channel 80. The bottom segment 122 of the pump housing 112 defines an intake slot 181 through which the intake 121 of the pump 114 extends.

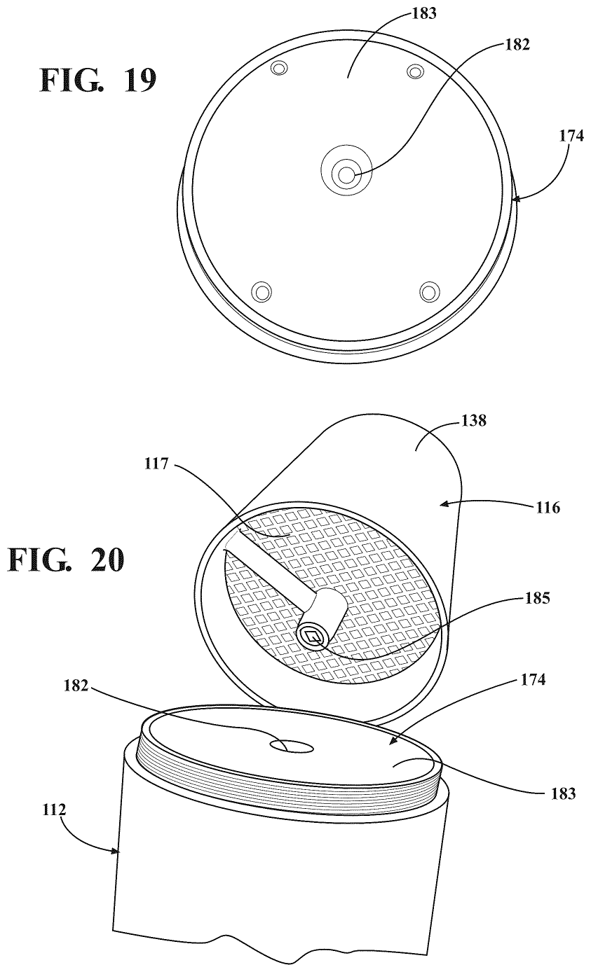

As best shown in FIGS. 18-20, the pump cap 174 is coupled to the top 138 of the pump housing 112 via a sealed interference fit along a perimeter of the pump cap 174. The pump cap 174 could be connected to the pump housing 112 in other ways. The pump cap 174 also has a cap channel 182 which spans between a center of a top surface 183 of the pump cap 174 and a bottom surface 184 of the pump cap 174, into alignment with the flow channel 180 of the flange 179 of the pump housing 112 such that pressurized air from the pump 114 flows through the cap channel 182. As best shown in FIG. 19, the cap channel 182 is fluidly connected to the inlet connector 158 of the vessel 116 and may be fluidly connected to an air passage 156 to deliver air to a top of the vessel 116 and second hose 164 and air emitting device 120, as discussed above. Alternatively, the inlet connector 158 may pass directly to an air emitting device 120 at the base 134 of the vessel 116. A seal 196, such as an o-ring 196, may be located around the inlet connector 158 to create a seal around the inlet connector 158 in the gap channel 182.

As best shown in FIGS. 13-15 and 21-22, the filter housing 176 generally is tube shaped and extends axially between a top side 186 and a bottom side 187. A cylindrical filter 178 is received through an opening along the bottom side 187 of the filter housing 176. A bottom cap 190 closes the bottom of the filter housing 176 to secure the filter 178 in place in the filter housing 176. The bottom cap 190 may be sealingly connected to the bottom of the filter housing 176 via threads or the like. The top side 186 of the filter housing 176 is sealingly connected to the bottom surface 184 of the pump cap 174, e.g., via bolts. The top side 186 of the filter housing 176 further defines a filter outlet 194 that is aligned with the intake slot 181 of the pump housing 112 for passing filtered air to the intake 121 of the pump 114. A pump hose 195 may also directly and seaslingly connect the filter outlet 194 to the intake 121 of the pump 114.The filter housing 176 further defines a side intake 192 for receiving air into the filter 178. A fan 193 (schematically shown) may also contained in the filter housing 76 for drawing air through the side intake 192 and passing it through the filter 178 and to the intake 121 of the pump 114 to provide filtered air to the pump 114. Alternatively, suction from the pump 114 alone may cause this passage of air through the filter 178 housing 176.

The modular construction of the second embodiment device 110 provides robust sealing against contaminants while enabling convenient maintenance and access to internal components. In particular, the pump 114 is protected within the pump housing 112, which is detachably connectable to the vessel 116 via the threaded connection (or other style connection) between the top end 115 and the lower end 117, thereby allowing the pump housing 112 to be removed for inspection, repair, or replacement without disturbing the vessel 116. The pump cap 174 further seals the top 138 of the pump housing 112 via a sealed interference fit (or other style connection), while still permitting disassembly when servicing is required, and directs pressurized air from the outlet 152 through the flow channel 80 and cap channel 182 toward the inlet connector 158. In addition, the filter housing 176 and filter 178 cooperate to supply filtered intake air to the intake 121 via the filter outlet 194 aligned with the intake slot 181, with the bottom cap 190 enabling rapid filter replacement through the bottom side 187. By filtering incoming air through the filter 178 (optionally assisted by fan 193 drawing air through side intake 192), cleaning gasses are inhibited from entering and interfering with operation of the pump 114, thereby improving reliability and service life in chemically aggressive environments.

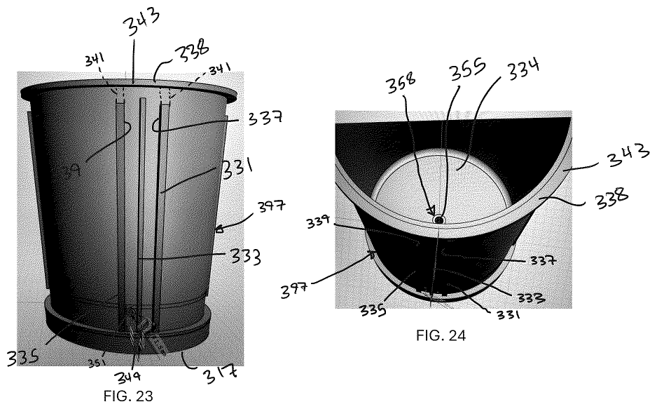

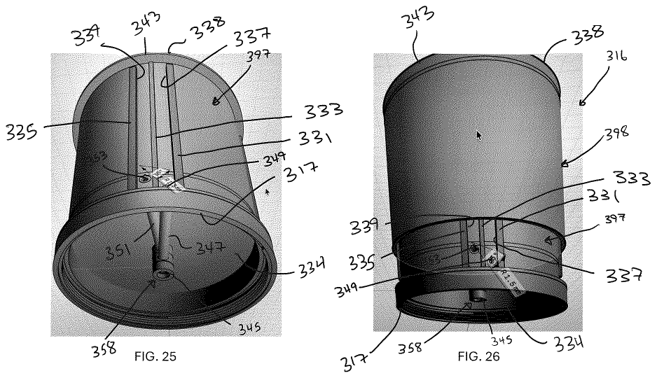

FIGS. 23-26 illustrate a vessel 316 of a third embodiment of the device for emitting cleaning gasses. The vessel 316 may effectively be used as a substitute for the vessels 116, 216 of either of the previously described embodiments and may rely on similar connections to the pump 14, 114 and pump housing 12, 112 and lid 18, 118.

According to this embodiment, the vessel 316 has an inner sleeve 397 and an outer sleeve 398 which is configured to slidingly fit about the inner sleeve 397. A base 334 is located along a lower end 317 of the inner sleeve 397. The inner sleeve 397 is configured to sealingly connect to a pump housing in the same manners as previously described (e.g., via threads). An inlet connector 358 protrudes from a bottom surface of the base 334 and defines an inlet orifice 345 at its end. As with previously described embodiments, the inlet connector 358 is connected to an outlet of the pump to permit the pump to pass pressurized air into the inlet connector 358 such that it can be conveyed into the chamber 340 of the housing 316.

An outer surface of the inner sleeve 397 has a first longitudinal rail 331, a second longitudinal rail 333 and a third longitudinal rail 335 which are all arranged in spaced and parallel relationship with one another and extend from the lower end 317 of the inner sleeve 397 to a location adjacent to an upper end 338 of the inner sleeve 397. A first channel 337 is defined between the first and second longitudinal rails 333, 335, and a second channel 339 is defined between the second and third longitudinal rails 335, 335. When the outer sleeve 398 is slid about the inner sleeve 397, the outer sleeve 398 sealingly engages a radially outer surface of each of the longitudinal rails 331, 333, 335, thus preventing air from passing between the longitudinal rails 331, 333, 335 and the outer sleeve 398. Furthermore, the outer sleeve 398 has a pair of notches 341 which effectively extend the first and third longitudinal rails 331, 335 to the upper end 338 of the inner sleeve 397 to prevent air from passing circumferentially outwardly beyond the channels 337, 339. The first and third longitudinal rails 331, 335 could alternatively extend to a rim 343 at the upper end 338 to accomplish this purpose. The second longitudinal rail 333 terminates short of the upper end 338 of the inner sleeve 397 such that the first and second channels 337, 339 are fluidly connected at their tops.

The inlet connector 358 includes an entry line 347 that is fluidly connected to the inlet orifice 345 and extends radially outwardly to a first channel orifice 349 at a bottom of the first channel 337 such that the entry line 347 directs pressurized air to the first channel 337. The inlet connector 358 also has an exit line 351 that extends from an exit orifice 353 at a location near a bottom of the second channel 339, radially to a center of the base 334 and axially upwardly to an outlet orifice 355 in the chamber 340 of the vessel 316. An air emitting device 320 may be located in the chamber 340 and connected to the outlet orifice 355 to emit pressurized air received from the pump 314.

Accordingly, during use, pressurized air passes from the pump, upwardly through the first channel 337, then downwardly through the second channel 339 and into the chamber 340 and air emitting device 320 via the outlet orifice 355. Routing pressurized air in this manner keeps air flow completely internal, while not allowing liquids from the chamber 10 to syphon into the pump.

Obviously, many modifications and variations of the present invention are possible in light of the above teachings and may be practiced otherwise than as specifically described while within the scope of the appended claims. These antecedent recitations should be interpreted to cover any combination in which the inventive novelty exercises its utility.

Clearly, changes may be made to what is described and illustrated herein without, however, departing from the scope defined in the accompanying claims. The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in any embodiment, even if not specifically shown or described. The same may also be varied in many ways. Such variations are not to be regarded as a departure from the disclosure, and all such modifications are intended to be included within the scope of the disclosure.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A device for emitting cleaning gasses into a space, comprising:
a vessel extending about and along an axis and having a sidewall extending between a lower end and an upper end and having a base adjacent to the lower end and defining a chamber;
a pump configured to emit a pressurized air; and
an air emitting device located in the chamber of the vessel adjacent to the base and fluidly connected to the pump for emitting pressurized air from the pump into the chamber to aerate a cleaning chemical in the chamber to release cleaning gasses into the space.

2. The device of claim 1, wherein the pump is positioned beneath the base of the vessel.

3. The device of claim 2, further including a pump housing located beneath the base of the vessel and sealed from the chamber of the vessel, and wherein the pump is located in the pump housing.

4. The device of claim 3, further including a filter housing positioned beneath, and coupled to the pump housing, and containing a filter configured to filter air prior to entering the pump.

5. The device of claim 4, wherein the filter housing is detachably connected to a bottom segment of the pump housing.

6. The device of claim 5, wherein the filter housing defines an opening at a bottom side of the filter housing, and wherein the filter is removably received by the opening at the bottom side of the filter housing.

7. The device of claim 6, further including a bottom cap removably closing the opening at the bottom side of the filter housing to hold the filter in the filter in the filter housing.

8. The device of claim 3, wherein the sidewall defines an air passage extending from an inlet in the pump housing and connected to the pump to an outlet adjacent to the upper end of the vessel, and wherein the air emitting device is connected to the outlet such that air travels through the pump, through the inlet, out of the outlet and to the air emitting device.

9. The device of claim 8, wherein a hose connects the outlet to the air emitting device, and wherein the hose extends along a length of the sidewall of the vessel.

10. The device of claim 3, wherein the pump housing is detachably connectable to the lower end of the vessel.

11. The device of claim 10, wherein the pump housing includes a plurality of tabs, and wherein the lower end of the vessel defines a plurality of slots configured to receive to tabs to releasably couple the vessel to the pump housing via relative rotation.

12. The device of claim 1, wherein the air emitting device comprises an air stone.

13. The device of claim 1, further comprising a lid coupled to the upper end of the vessel and defining an array of orifices sized and arranged to permit cleaning gasses to escape from the chamber while inhibiting liquid splashes.

14. The device of claim 1, wherein a pump cap removeably closes and seals a top of the pump housing between the pump housing and the base of the vessel.

15. The device of claim 14, wherein a cap channel extends axially through the cap to fluidly connect the pump to the air emitting device.

16. A method for emitting cleaning gasses into a space, comprising:
positioning a vessel in the space, the vessel extending about and along an axis and having a base and a sidewall extending between a lower end and an upper end and defining a chamber;
placing a cleaning chemical in the chamber; and
operating a pump to deliver pressurized air through an air emitting device located in the chamber adjacent to the base to aerate the cleaning chemical and release cleaning gasses into the space.

17. The method of claim 16, wherein operating the pump comprises operating the pump while the cleaning chemical includes a tablet dissolving in liquid within the chamber.

18. The method of claim 16, wherein operating the pump comprises delivering the pressurized air from the pump through an air passage defined in the sidewall and extending from an inlet adjacent the base to an outlet adjacent the upper end, and further directing the pressurized air from the outlet through a hose to the air emitting device.

19. The method of claim 16, further comprising filtering intake air prior to entering the pump with a filter disposed in a filter housing coupled to a pump housing in which the pump is disposed.

20. The method of claim 16, wherein the air emitting device comprises an air stone disposed adjacent to the base of the vessel.
